(19) United States
(12) Patent Application Publication
(10) Pub. No.: US 2005/0063929 A1
(43) Pub. Date: Mar. 24, 2005

(54) Cosmetic hair composition comprising at least one tridecyl trimellitate and at least one isoeicosane

(76) Inventor: Sabine Vrignaud, La Plaine St Denis (FR)

Correspondence Address: Thomas L. Irving;FINNEGAN, HENDERSON, FARABOW,, GARRETT & DUNNER, L.L.P., 1300 I Street, N.W., Washington, DC, 20005-3315, US

(21) Appl. No.: 10/868,009

(22) Filed: Jun. 16, 2004

Related U.S. Application Data
(60) Provisional application No. 60/505,165, filed on Sep. 24, 2003.

(30) Foreign Application Priority Data
Jun. 17, 2003 (FR) 0307291

(51) Int. Cl. A61K 7/06

(52) U.S. Cl. 424/70.1

(57) ABSTRACT

The invention relates to a cosmetic hair composition comprising, in a cosmetically acceptable medium, at least one tridecyl trimellitate and at least one isoeicosane. It is also directed towards processes, including process for treating the hairstyle using the composition, and a process for giving the hair sheen.

This application claims benefit of U.S. Provisional Application No. 60/505,165, filed Sep. 24, 2003.

The present disclosure relates to novel cosmetic hair compositions comprising at least one tridecyl trimellitate and at least one isoeicosane. It is also directed towards processes, for example, a process for treating the hair by applying the compositions, and also a process for giving the hair sheen by applying the compositions.

The use of oils of the benzoic triacid triester type, such as tridecyl trimellitate, is well known for the formulation of numerous cosmetic products comprising a fatty phase.

It is described in patent application EP-A-0 792 637 for the preparation of lipsticks. It is also mentioned in patent application EP-A-0 194 055 for the preparation of anhydrous cosmetic formulations free of mineral oil. In U.S. Pat. No. 4,940,577, this type of oil constitutes the fatty phase of transparent microemulsions of the water-in-oil type with a low water content, which may be used as antisun products.

There is a need for hair products that can give the hair long-lasting sheen, without making the hair look too greasy. This need is not addressed by the compositions of the prior art.

In one embodiment, the Applicant has discovered, surprisingly, that compositions comprising at least one tridecyl trimellitate and at least one isoeicosane provide the hair with at least one of strong, long-lasting sheen and a smooth feel.

The Applicant has also found that in some embodiments these compositions adhere well to the hair and have good cosmetic properties.

The Applicant has also discovered that these compositions, in some embodiments, can be formulated in an alcoholic medium.

One non-limiting embodiment of the present disclosure is a cosmetic hair composition, wherein the composition comprises, in a cosmetically acceptable medium, at least one tridecyl trimellitate and at least one isoeicosane.

Another subject of the invention relates to a cosmetic hair process, such as a process for treating the hair using the compositions disclosed herein.

A subject of the invention is also a process for giving the hair sheen by applying the compositions disclosed herein to the hair.

Tridecyl trimellitate is an oil having the following formula:

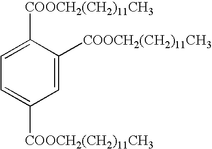

The commercial products sold under the name Dub TMTD by the company Stearineries Dubois and under the name Liponate TDTM by the company Lipo Chemicals may be used.
lsoeicosane is a hydrocarbon that is liquid at room temperature. The commercial product sold under the name Permethyl 102 by the company Bayer may, for example, be used as the isoeicosane disclosed herein.

In one embodiment, the individual concentrations of tridecyl trimellitate and of isoeicosane range from 0.01% to 15%, such as from 1% to 10% by weight relative to the total weight of the composition.

In a further embodiment, the mixture comprising at least one tridecyl trimellitate and at least one isoeicosane is present in the composition in an amount ranging from 0.05% to 20% by weight relative to the total weight of the composition, such as ranging from 5% to 10% by weight relative to the total weight of the composition.

The tridecyl trimellitate/isoeicosane weight ratio, may, for example, range from 100 to 0.01, such as from 10 to 0.1. The ratio may range, for further example, from 0.7 to 1.3.

The cosmetically acceptable medium comprises, for example, water or at least one cosmetically acceptable solvent such as a solvent chosen from alcohols and water-solvent(s) mixtures. These solvents may be, for example, C₁-C₄ alcohols.

Among these alcohols, mention may be made of ethanol and isopropanol. In one non-limiting embodiment ethanol may be used.

The composition may also comprise at least one fixing polymer.

For the purposes of the present disclosure, the term “fixing polymer” means any polymer that can give the hair a shape or that can maintain this shape.

The at least one fixing polymer may be chosen from cationic, anionic, amphoteric and nonionic polymers and mixtures thereof. In one embodiment, the at least one fixing polymer is a mixture of fixing polymers.

The cationic fixing polymers that may be used according to the present disclosure may be chosen from, for example, polymers comprising primary, secondary, tertiary and quaternary amine groups forming part of the polymer chain or directly attached thereto, and having a molecular weight ranging from 500 to about 5,000,000, for example, 1,000 to 3,000,000.

Among these polymers, mention may be made, for example, of the following cationic polymers:
(1) homopolymers or copolymers derived from acrylic or methacrylic esters or amides with amine functions, and comprising at least one of the units of the following formulae: 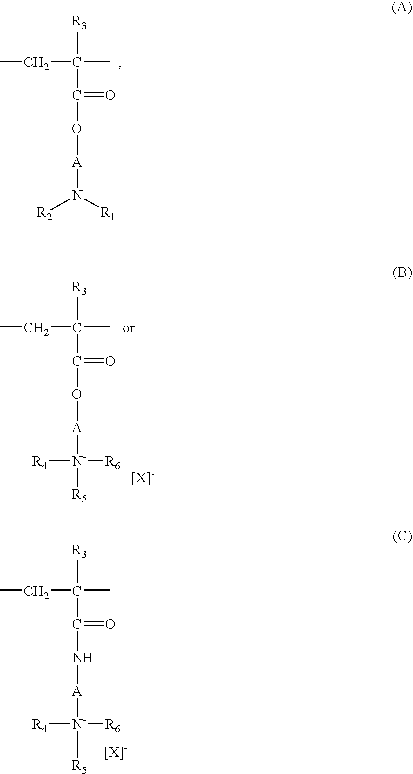
   wherein:
R₁ and R₂, which may be identical or different, are chosen from hydrogen atoms and alkyl groups having from 1 to 6 carbon atoms;
R₃ is chosen from hydrogen atoms and CH₃ groups;
A is chosen from linear and branched alkyl groups comprising from 1 to 6 carbon atoms and linear and branched hydroxyalkyl groups comprising from 1 to 4 carbon atoms;
R₄, R₅ and R₆, which may be identical or different, are chosen from alkyl groups having from 1 to 18 carbon atoms and benzyl groups;
X is chosen from methosulfate anions and halides such as chloride or bromide.

The copolymers of family (1) may also comprise at least one comonomer unit which can be chosen from the family of acrylamides, methacrylamides, diacetone acrylamides, acrylamides and methacrylamides substituted on the nitrogen with lower alkyl groups, groups derived from acrylic or methacrylic acids or esters thereof; vinyllactams such as vinylpyrrolidone or vinylcaprolactam; and vinyl esters.

Thus, among these copolymers of family (1), non-limiting mention may be made of:
copolymers of acrylamide and dimethylaminoethyl methacrylate quaternized with at least one of dimethyl sulfate and dimethyl halide, such as the one sold under the name Hercofloc® by the company Hercules,
copolymers of acrylamide and of methacryloyloxyethyltrimethylammonium chloride, described, for example, in patent application EP-A-080 976 and sold under the name Bina Quat P 100 by the company Ciba Geigy,
copolymers of acrylamide and of methacryloyloxyethyltrimethylammonium methosulfate, such as the product sold under the name Reten by the company Hercules,
quaternized or non-quaternized vinylpyrrolidone/dialkylaminoalkyl acrylate and methacrylate copolymers, such as the products sold under the name “Gafquat®” by the company ISP, such as, for example, “Gafquat® 734” or “Gafquat® 755” or alternatively the products known as “Copolymer® 845, 958 and 937”. These polymers are described in detail in French patents 2 077 143 and 2 393 573,
fatty-chain polymers comprising at least one vinylpyrrolidone unit, such as the products sold under the name Styleze W20 and Styleze W10 by the company ISP,
dimethylaminoethyl methacrylate/vinylcaprolactam/vinylpyrrolidone terpolymers, such as the product sold under the name Gaffix® VC 713 by the company ISP, and
quaternized vinylpyrrolidone/dimethylaminopropylmethacrylamide copolymers, such as the product sold under the name “Gafquat® HS 100” by the company ISP;
(2) cationic polysaccharides, for example, those comprising quaternary ammonium, such as those described in U.S. Pat. Nos. 3,589,578 and 4,031,307, such as guar gums comprising trialkylammonium cationic groups. Such products are sold, for example, under the trade names Jaguar C13S, Jaguar C15 and Jaguar C17 by the company Meyhall;
(3) quaternary copolymers of vinylpyrrolidone and of vinylimidazole;
(4) chitosans or salts thereof; the salts that can be used are, for example, chitosan acetate, lactate, glutamate, gluconate and pyrrolidonecarboxylate. Among these compounds, non-limiting mention may be made of chitosan having a degree of deacetylation of 90.5% by weight, sold under the name Kytan Brut Standard by the company Aber Technologies, and chitosan pyrrolidonecarboxylate sold under the name Kytamer® PC by the company Amerchol.

(5) cationic cellulose derivatives such as copolymers of cellulose and of cellulose derivatives grafted with a water-soluble monomer comprising a quaternary ammonium, and disclosed, for example, in U.S. Pat. No. 4,131,576, such as hydroxyalkylcelluloses, for instance hydroxymethyl-, hydroxyethyl- and hydroxypropyl-celluloses grafted, for example, with at least one of a methacryloyloxyethyltrimethylammonium, methacrylamidopropyltrimethylammonium and a dimethyldiallylammonium salt.

The products sold corresponding to this definition are, for example, the products sold under the name “Celquat L 200” and “Celquat H 100” by the company National Starch.

The anionic fixing polymers may be chosen, for example, from polymers comprising groups derived from carboxylic acid, sulfonic acid or phosphoric acid and having a number average molecular weight approximately ranging from 500 to 5,000,000.

Examples of groups derived from carboxylic acids include unsaturated monocarboxylic or dicarboxylic acid monomers such as those corresponding to the formula: 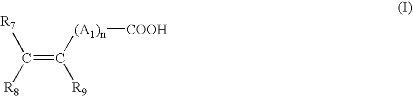
wherein n is an integer from 0 to 10, A₁ is a methylene group, optionally connected to the carbon atom of the unsaturated group, or to the neighbouring methylene group when n is greater than 1, via a hetero atom such as oxygen or sulfur, R₇ is a hydrogen atom or a phenyl or benzyl group, R₈ is a hydrogen atom or a lower alkyl or carboxyl group, R₉ denotes a hydrogen atom, a lower alkyl group or a —CH₂—COOH, phenyl or benzyl group.

In the above mentioned formula, a lower alkyl group may be a group having 1 to 4 carbon atoms such as methyl and ethyl.

The anionic fixing polymers comprising carboxylic groups disclosed herein may be, for example:
A) acrylic and methacrylic acid homo- or copolymers, or salts thereof such as the products sold under the names Versicol® E or K by Allied Colloid and Ultraholde by the BASF. The copolymers of acrylic acid and of acrylamide in the form of their sodium salts may be, for example, those sold under the names Reten 421, 423 or 425 by the company Hercules, as the sodium salts of polyhydroxycarboxylic acids
B) copolymers of acrylic or methacrylic acids with a monoethylenic monomer such as ethylene, styrene, vinyl esters, acrylic and methacrylic acid esters, optionally grafted onto a polyalkylene glycol such as polyethylene glycol and optionally crosslinked. Such polymers are described, for example, in French patent 1 222 944 and German patent application 2 330 956, the copolymers of this type comprising an optionally N-alkylated and/or hydroxyalkylated acrylamide unit in their chain such as those described in Luxembourg patent applications 75370 and 75371 or those sold under the name Quadramer by the company American Cyanamid. Non-limiting mention may also be made of copolymers of acrylic acid and of a C₁-C₄ alkyl methacrylate and terpolymers of vinylpyrrolidone, acrylic acid and a C₁-C₂₀ alkyl methacrylate, for example lauryl methacrylate, such as the product sold by the company ISP under the name Acrylidone® LM and methacrylic acid/ethyl acrylate/tert-butyl acrylate terpolymers such as the product sold under the name Luvimer® 100 P by the company BASF. Non-limiting mention may also be made of methacrylic acid/acrylic acid/ethyl acrylate/methyl acrylate copolymers as an aqueous dispersion, sold under the name Amerholde DR 25 by the company Amerchol.
C) crotonic acid copolymers, such as those comprising at least one unit chosen from vinyl acetate and propionate in their chain and optionally at least one other monomer such as those chosen from allylic esters, methallylic esters, vinyl ethers and vinyl esters of linear and branched saturated carboxylic acids with a long hydrocarbon chain such as those comprising at least 5 carbon atoms, it being possible for these polymers optionally to be grafted or crosslinked, or alternatively a vinyl, allylic or methallylic ester of an α- or β-cyclic carboxylic acid. Such polymers are described, for example, in French patents 1 222 944, 1 580 545, 2 265 782, 2 265 781, 1 564 110 and 2 439 798. Commercial products falling into this class are the resins 28-29-30, 26-13-14 and 28-13-10 sold by the company National Starch.
D) copolymers derived from C₄-C₈ monounsaturated carboxylic acids or anhydrides chosen from:
copolymers comprising (i) at least one of maleic, fumaric and itaconic acids and anhydrides and (ii) at least one monomer chosen from vinyl esters, vinyl ethers, vinyl halides, phenylvinyl derivatives, acrylic acids and their esters, the anhydride functions of these copolymers optionally being monoesterified or monoamidated. Such polymers are described, for example, in U.S. Pat. Nos. 2,047,398, 2,723,248 and 2,102,113 and GB patent GB 839 805 such as those sold under the names Gantreze AN or ES by the company ISP,
copolymers comprising (i) at least one maleic, citraconic and itaconic anhydride unit and (ii) at least one monomer chosen from allylic and methallylic esters optionally comprising one or more acrylamide, methacrylamide, α-olefin, acrylic and methacrylic ester, acrylic and methacrylic acid, and vinylpyrrolidone group in their chain,
   the anhydride functions of these copolymers optionally being monoesterified or monoamidated.

These polymers are described, for example, in French patents FR 2 350 384 and FR 2 357 241 by the Applicant.
E) polyacrylamides comprising carboxylate groups.

The homopolymers and copolymers comprising sulfonic groups are polymers comprising at least one unit chosen from vinylsulfonic, styrenesulfonic, naphthalenesulfonic and acrylamidoalkylsulfonic units.

These polymers can be chosen, for example, from:
polyvinylsulfonic acid salts having a molecular weight from approximately 1,000 to 100,000, as well as the copolymers with an unsaturated comonomer such as acrylic or methacrylic acids and their esters, as well as acrylamide or its derivatives, vinyl ethers and vinylpyrrolidone;
polystyrenesulfonic acid salts such as the sodium salts which are sold under the names Flexan® 500 and Flexan® 130 by National Starch. These compounds are described in patent FR 2 198 719;
polyacrylamidesulfonic acid salts, such as those mentioned in U.S. Pat. No. 4,128,631, for example, polyacrylamidoethylpropanesulfonic acid sold under the name Cosmedia Polymer HSP 1180 by Henkel.

As another anionic fixing polymer that can be used according to the disclosure, non-limiting mention may be made of the branched block anionic polymer sold under the name Fixate G100 by the company Noveon.

According to the disclosure, the anionic fixing polymers may be chosen from, for example, acrylic acid copolymers, such as the acrylic acid/ethyl acrylate/N-tert-butylacrylamide terpolymers sold under the name Ultraholde Strong by the company BASF; copolymers derived from crotonic acid, such as vinyl acetate/vinyl tert-butylbenzoate/crotonic acid terpolymers and the crotonic acid/vinyl acetate/vinyl neododecanoate terpolymers sold under the name Resin 28-29-30 by the company National Starch; polymers derived from maleic, fumaric or itaconic acids or anhydrides with vinyl esters, vinyl ethers, vinyl halides, phenylvinyl derivatives and acrylic acid and esters thereof, such as the methyl vinyl ether/monoesterified maleic anhydride copolymers sold under the name Gantrez® by the company ISP; the copolymers of methacrylic acid and of methyl methacrylate sold under the name Eudragit® L by the company Rohm Pharma; the copolymers of methacrylic acid and of ethyl acrylate sold under the name Luvimer® MAEX or MAE by the company BASF; the vinyl acetate/crotonic acid copolymers sold under the name Luviset CA 66 by the company BASF; the vinyl acetate/crotonic acid copolymers grafted with polyethylene glycol sold under the name Aristoflex A® by the company BASF; and the polymer sold under the name Fixate G100 by the company Noveon.

Among the anionic fixing polymers mentioned above, non-limiting mention may be made of the methyl vinyl ether/monoesterified maleic anhydride copolymer sold under the name Gantrez® ES 425 by the company ISP, the acrylic acid/ethyl acrylate/N-tert-butylacrylamide terpolymers sold under the name Ultrahold® Strong by the company BASF, the copolymers of methacrylic acid and of methyl methacrylate sold under the name Eudragit® L by the company Rohm Pharma, the vinyl acetate/vinyl tert-butylbenzoate/crotonic acid terpolymers and the crotonic acid/vinyl acetate/vinyl neododecanoate terpolymers sold under the name Resin 28-29-30 by the company National Starch, the copolymers of methacrylic acid and of ethyl acrylate sold under the name Luvimer® MAEX or MAE by the company BASF, the vinylpyrrolidone/acrylic acid/lauryl methacrylate terpolymer sold under the name Acrylidone LM by the company ISP, and the polymer sold under the name Fixate G100 by the company Noveon.

The amphoteric fixing polymers that can be used in accordance with the disclosure can be chosen, for example, from polymers comprising units B and C distributed randomly in the polymer chain, wherein B is a unit derived from a monomer comprising at least one basic nitrogen atom and C is a unit derived from an acid monomer comprising at least one group chosen from carboxylic and sulfonic groups, or alternatively B and C can be groups chosen from those derived from carboxybetaine and sulfobetaine zwitterionic monomers;
B and C can also be a cationic polymer chain comprising primary, secondary, tertiary or quaternary amine groups, wherein at least one of the amine groups bears a carboxylic or sulfonic group connected via a hydrocarbon group or alternatively B and C form part of a chain of a polymer comprising an a, β-dicarboxylic ethylene unit in which one of the carboxylic groups has been made to react with a polyamine comprising at least one primary or secondary amine groups.

The amphoteric fixing polymers corresponding to the definition given above may also be chosen from the following polymers:
(1) copolymers having acidic vinyl and basic vinyl units, such as those resulting from the copolymerization of a monomer derived from a vinyl compound bearing a carboxylic group such as acrylic acid, methacrylic acid, maleic acid, α-chloroacrylic acid, and a basic monomer derived from a substituted vinyl compound comprising at least one basic atom, such as dialkylaminoalkyl methacrylate and acrylate, dialkylaminoalkylmethacrylamides and -acrylamides. Such compounds are described in U.S. Pat. No. 3,836,537.
(2) polymers comprising units derived from:
   a) at least one monomer chosen from acrylamides and methacrylamides substituted on the nitrogen with an alkyl radical,
   b) at least one acidic comonomer comprising at least one reactive carboxylic group, and
   c) at least one basic comonomer such as esters containing primary, secondary, tertiary and quaternary amine substituents of acrylic and methacrylic acids and the product of quaternization of dimethylaminoethyl methacrylate with dimethyl or diethyl sulfate.

The N-substituted acrylamides or methacrylamides according to the disclosure may be, for example, compounds wherein the alkyl groups comprise from 2 to 12 carbon atoms such as N-ethylacrylamide, N-tert-butylacrylamide, N-tert-octylacrylamide, N-octylacrylamide, N-decylacrylamide, N-dodecylacrylamide and the corresponding methacrylamides.

The acidic comonomers may be chosen from, for example, acrylic acid, methacrylic acid, crotonic acid, itaconic acid, maleic acid and fumaric acid and alkyl monoesters, having from 1 to 4 carbon atoms, of maleic or fumaric acids or anhydrides.

Non-limiting examples of the basic comonomers mentioned above are aminoethyl, butylaminoethyl, N,N′-dimethylaminoethyl and N-tert-butylaminoethyl methacrylates.

The copolymers whose CTFA (4th edition, 1991) name is octylacrylamide/acrylates/butylaminoethyl methacrylate copolymer, such as the products sold under the name Amphomer or Lovocryl 47 by the company National Starch, may be used.
(3) crosslinked and acylated polyamino amides partially or totally derived from polyamino amides of general formula (II): 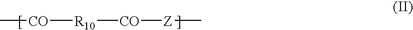
   wherein R₁₀ is a divalent group derived from a saturated dicarboxylic acid, a mono- or dicarboxylic aliphatic acid comprising an ethylenic double bond, an ester of a lower alkanol, having 1 to 6 carbon atoms, of these acids, or a group derived from the addition of any one of said acids to a bis(primary) or bis(secondary) amine; and Z is a group derived from a bis(primary), mono- or bis(secondary) polyalkylene-polyamine and may represent:
a) in proportions ranging from 60 to 100 mol % of the group (IV): 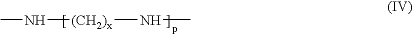
   wherein x=2 and p=2 or 3, or alternatively x=3 and p=2,
   this group being derived from diethylenetriamine, triethylenetetraamine or dipropylenetriamine;
b) in proportions ranging from 0 to 40 mol % of the group (IV) above wherein x=2 and p=1 and which is derived from ethylenediamine, or the group derived from piperazine: 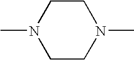
c) in proportions ranging from 0 to 20 mol %, the —NH(CH₂)₆—NH— group being derived from hexamethylenediamine, these polyamino amides being crosslinked by addition of a difunctional crosslinking agent chosen from epihalohydrins, diepoxides, dianhydrides and bis-unsaturated derivatives, using from 0.025 to 0.35 mol of crosslinking agent per amine group of the polyamino amide and acylated by the action of acrylic acid, chloroacetic acid or an alkane sultone, or salts thereof.

The saturated carboxylic acids may be chosen, for example, from acids having 6 to 10 carbon atoms, such as adipic acid, 2,2,4-trimethyladipic acid, 2,4,4-trimethyladipic acid, terephthalic acid, acids comprising an ethylenic double bond such as, for example, acrylic acid, methacrylic acid and itaconic acid.

The alkane sultones used in the acylation may be, for example, propane sultone or butane sultone, the salts of the alkylating agents may be, for example, sodium or potassium salts.
(4) polymers comprising zwitterionic units of formula: 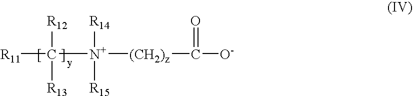
   wherein R₁₁ is a polymerizable unsaturated group such as an acrylate, methacrylate, acrylamide or methacrylamide group, y and z represent an integer from 1 to 3, R₁₂ and R₁₃, which may be identical or different, are chosen from hydrogen atoms, methyl groups, ethyl groups, and propyl groups, R₁₄ and R₁₅, which may be identical or different, are chosen from hydrogen atoms and alkyl groups such that the sum of the carbon atoms in R₁₄ and R₁₅ does not exceed 10.

The polymers comprising such units can also comprise units derived from nonzwitterionic monomers such as dimethyl or diethylaminoethyl acrylate or methacrylate or alkyl acrylates or methacrylates, acrylamides or methacrylamides or vinyl acetate.

By way of example, mention may be made of the copolymers of methyl methacrylate/methyl dimethylcarboxymethylammonioethyl methacrylate such as the product sold under the name Diaformer Z301 by the company Sandoz.
(5) polymers derived from chitosan comprising monomer units corresponding to the following formulae: 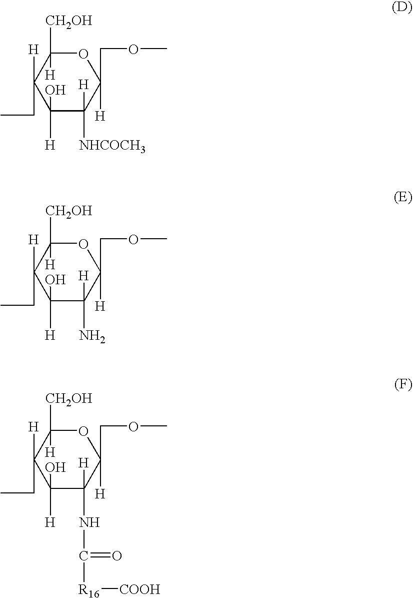
   the unit (D) being present in proportions ranging from 0 to 30%, the unit (E) in proportions ranging from 5 to 50% and the unit (F) in proportions ranging from 30 to 90%, it being understood that, in this unit (F), R₁₆ represents a group of formula: 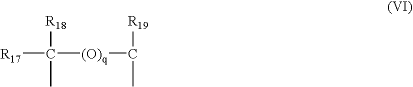
   (VI) wherein, if q=0, R₁₇, R₁₈ and R₁₉, which may be identical or different, are chosen from hydrogen atoms, methyls, hydroxyls, acetoxy and amino residues, monoalkylamine residues, and dialkylamine residues which are optionally interrupted by one or more nitrogen atoms and/or optionally substituted with one or more amine, hydroxyl, carboxyl, alkylthio or sulfonic groups, an alkylthio residue in which the alkyl group bears an amino residue, at least one of the groups R₁₇, R₁₈ and R₁₉ being, in this case, a hydrogen atom;
or, if q=1, R₁₇, R₁₈ and R₁₉ each represent a hydrogen atom, as well as the salts formed by these compounds with bases or acids.
(6) polymers corresponding to the general formula (V) which are described, for example, in French patent 1 400 366: 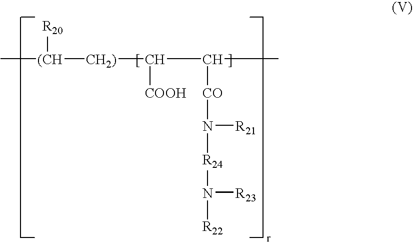
   wherein R₂₀ represents a hydrogen atom, a CH₃O, CH₃CH₂O or a phenyl group; R₂₁ is a hydrogen atom or a lower alkyl group such as methyl or ethyl; R₂₂ is a hydrogen atom or a C₁₋₆ lower alkyl group such as methyl or ethyl; and R₂₃ is a C₁₋₆ lower alkyl group such as methyl or ethyl or a group corresponding to the formula: —R₂₄—N(R₂₂)₂, R₂₄ representing a —CH₂—CH₂—, —CH₂—CH₂—CH₂— or —CH₂—CH(CH₃)— group, R₂₂ having the meanings mentioned above,
(7) polymers derived from the N-carboxyalkylation of chitosan, such as N-carboxymethylchitosan or N-carboxybutylchitosan sold under the name “Evalsan” by the company Jan Dekker.
(8) amphoteric polymers of the type -D-X-D-X— chosen from:
a) polymers obtained by the reaction of chloroacetic acid or sodium chloroacetate with compounds comprising at least one unit of formula:
   -D-X-D-X-D- (VI)
   where D denotes a group 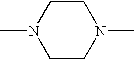
   and X denotes the symbol E or E′, E or E′, which may be identical or different, denote a divalent group which is an alkylene group with a straight or branched chain containing up to 7 carbon atoms in the main chain, which is unsubstituted or substituted with hydroxyl groups and which can comprise, in addition to oxygen, nitrogen and sulfur atoms, 1 to 3 aromatic and/or heterocyclic rings; the oxygen, nitrogen and sulfur atoms being present in the form of ether, thioether, sulfoxide, sulfone, sulfonium, alkylamine or alkenylamine groups, hydroxyl, benzylamine, amine oxide, quaternary ammonium, amide, imide, alcohol, ester and/or urethane groups.
b) polymers of formula:
   -D-X-D-X— (VI′)
   where D denotes a group 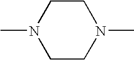
   and X denotes the symbol E or E′, wherein at least one X is E′; E having the meaning given above and E′ is a divalent group which is an alkylene group with a straight or branched chain having up to 7 carbon atoms in the main chain, which is unsubstituted or substituted with one or more hydroxyl groups and containing one or more nitrogen atoms, the nitrogen atom being substituted with an alkyl chain which is optionally interrupted by an oxygen atom and necessarily comprising one or more carboxyl functions or one or more hydroxyl functions and betainized by reaction with chloroacetic acid or sodium chloroacetate.
(9) (C₁-C₅)alkyl vinyl ether/maleic anhydride copolymers partially modified by semiamidation with an N,N-dialkylaminoalkylamine such as N,N-dimethylaminopropylamine or by semiesterification with an N,N-dialkanolamine. These copolymers can also comprise other vinyl comonomers such as vinylcaprolactam.

Among the amphoteric fixing polymers described above, non-limiting mention may be made of those of family (3), such as the copolymers whose CTFA name is octylacrylamide/acrylates/butylaminoethyl methacrylate copolymer, such as the products sold under the names Amphomer®, Amphomer® LV 71 or Lovocryl® 47 by the company National Starch; and those of family (4) such as the copolymers of methyl methacrylate/methyl dimethylcarboxymethylammonioethyl methacrylate, sold, for example, under the name Diaformer® Z301 by the company Sandoz.

The nonionic fixing polymers disclosed herein are chosen, for example, from:
polyalkyloxazolines;
vinyl acetate homopolymers;
vinyl acetate copolymers, for instance copolymers of vinyl acetate and of acrylic ester; copolymers of vinyl acetate and of ethylene; copolymers of vinyl acetate and of maleic ester, for example dibutyl maleate;
homopolymers and copolymers of acrylic esters, for instance copolymers of alkyl acrylates and of alkyl methacrylates, such as the products sold by the company Rohm & Haas under the names Primal® AC-261 K and Eudragit® NE 30 D, by the company BASF under the name 8845, or by the company Hoechst under the name Appretan® N9212;
copolymers of acrylonitrile and of a nonionic monomer chosen, for example, from butadiene and alkyl (meth)acrylates; mention may be made of the products sold under the name CJ 0601 B by the company Rohm & Haas;
styrene homopolymers;
styrene copolymers, for instance copolymers of styrene and of an alkyl (meth)acrylate, such as the products Mowilith® LDM 6911, Mowilith® DM 611 and Mowilith® LDM 6070 sold by the company Hoechst, and the products Rhodopas® SD 215 and Rhodopas® DS 910 sold by the company Rh6ne-Poulenc; copolymers of styrene, of alkyl methacrylate and of alkyl acrylate; copolymers of styrene and of butadiene; or copolymers of styrene, of butadiene and of vinylpyridine;
polyamides;
vinyllactam homopolymers other than vinylpyrrolidone homopolymers, such as the polyvinylcaprolactam sold under the name Luviskol® Plus by the company BASF; and
vinyllactam copolymers such as a poly(vinylpyrrolidone/vinyllactam) copolymer sold under the trade name Luvitec® VPC 55K65W by the company BASF, poly(vinylpyrrolidone/vinyl acetate) copolymers, such as those sold under the name PVPVA® S630L by the company ISP, Luviskol® VA 73, VA 64, VA 55, VA 37 and VA 28 by the company BASF; and poly(vinylpyrrolidone/vinyl acetate/vinyl propionate) terpolymers, for instance the product sold under the name Luviskol® VAP 343 by the company BASF.

The alkyl groups of the nonionic polymers mentioned above contain, for example, from 1 to 6 carbon atoms.

According to the disclosure, it is also possible to use fixing polymers of grafted silicone type comprising a polysiloxane portion and a portion consisting of a nonsilicone organic chain, one of the two portions constituting the main chain of the polymer, and the other being grafted onto said main chain.

These polymers are described, for example, in patent applications EP-A-0 412 704, EP-A-0 412 707, EP-A-0 640 105 and WO 95/00578, EP-A-0 582 152 and WO 93/23009 and U.S. Pat. Nos. 4,693,935, 4,728,571 and 4,972,037.

These polymers may be amphoteric, anionic or nonionic, for example, anionic or nonionic.

Such polymers are, for example, copolymers that can be obtained by free radical polymerization from the monomer mixture formed from:
a) 50 to 90% by weight of tert-butyl acrylate;
b) 0 to 40% by weight of acrylic acid; and
c) 5 to 40% by weight of a silicone macromer of formula: 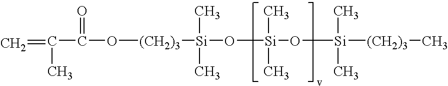
   wherein v is a number from 5 to 700; the weight percentages being calculated relative to the total weight of the monomers.

Other examples of grafted silicone polymers are, for example, polydimethylsiloxanes (PDMSs) onto which are grafted, via a thiopropylene-type connecting chain, mixed polymer units of the poly(meth)acrylic acid type and of the polyalkyl (meth)acrylate type; and polydimethylsiloxanes (PDMSs) onto which are grafted, via a thiopropylene-type connecting chain, polymer units of the polyisobutyl (meth)acrylate type.

Another type of silicone fixing polymer that may be mentioned is the product Luviflex® Silk, sold by the company BASF.

Functionalized or non-functionalized, silicone or non-silicone, cationic, nonionic, anionic or amphoteric polyurethanes and mixtures thereof may also be used as fixing polymers.

The polyurethanes intended by the present disclosure are, for example, those disclosed in patents EP 0 751 162, EP 0 637 600, EP 0 648 485 and FR 2 743 297, and patents EP 0 656 021 and WO 94/03510 from the company BASF and EP 0 619 111 from the company National Starch.

As polyurethanes that are suitable for the present disclosure, non-limiting mention may be made of the products sold under the names Luviset Pur® and Luviset® Si-P r by the company BASF.

In one embodiment, when the fixing polymers are present, the concentration of these fixing polymers ranges from 0.01% to 20%, for example, 0.05% to 15% such as from 0.1% to 10% by weight relative to the total weight of the composition.

The composition disclosed herein may further comprise at least one additive chosen from nonionic, anionic, cationic, amphoteric and zwitterionic surfactants, fragrances, screening agents, preserving agents, proteins, vitamins, nonionic, anionic, cationic, amphoteric and zwitterionic polymers other than the fixing polymers described above, mineral oils, plant and synthetic oils, thickeners and any other additive conventionally used in cosmetic compositions, such as antidandruff agents, agents for combating hair loss, electrolytes, dyes, pigments, moisturizers such as glycerol and other polyols, and reducing agents.

These additives are optionally present in the composition in proportions that may range, for example, from 0.001% to 20% by weight relative to the total weight of the composition. The precise amount of each additive depends on its nature and may readily be determined by a person skilled in the art, and will depend on the hair application selected.

Needless to say, a person skilled in the art will take care to select the additives such that the advantageous properties of the composition disclosed herein are not, or are not substantially, adversely affected by the envisaged addition.

The compositions of the invention may be used for manufacturing numerous hair products such as, for example, products for fixing and/or holding the hair, conditioning products, and haircare products.

These compositions may be packaged in various forms, for example, in pump-dispenser bottles or in aerosol cans, so as to be able to apply the composition in vaporized form or in mousse form. Such packaging forms are indicated, for example, when it is desired to obtain a spray, a lacquer or a mousse for fixing or treating the hair. The compositions disclosed herein may also be in the form of creams, gels, emulsions or lotions.

In one embodiment, when the compositions disclosed herein are packaged in aerosol form in order to obtain a lacquer or a mousse, they comprise at least one propellant that may be chosen from volatile hydrocarbons such as n-butane, propane, isobutane and pentane; halogenated hydrocarbons; and mixtures thereof. Carbon dioxide, nitrous oxide, dimethyl ether (DME), nitrogen and compressed air may also be used as propellant. Mixtures of propellants may also be used. Dimethyl ether, for example, may be used.

The propellant may be present, for example, in a concentration ranging from 5% to 90% by weight, for example, in a concentration ranging from 10% to 60% by weight relative to the total weight of the composition in the aerosol device.

The examples that follow are intended to illustrate the invention.

### EXAMPLE 1

Composition A below was applied to a 5-gram lock of natural European chestnut-brown hair.

Composition A:

| | |
|---|---|
| Liponate TDTM (1) | 3.5% |
| Permethyl 102 A (2) | 3.5% |
| Ethanol | qs 100% |

(1) tridecyl trimellitate sold by the company Lipo Chemicals
(2) isoeicosane sold by the company Bayer

Composition A was applied using a pump-dispenser bottle onto natural hair. After drying, it was observed that the lock is shiny, feels soft and looks pleasant.

### EXAMPLE 2

Composition B below was applied to a 5-gram lock of natural European chestnut-brown hair.

Composition B:

| | |
|---|---|
| Permethyl 102 A (1) | 5% |
| Liponate TDTM (2) | 5% |
| Pecosil PS-100 (3) | 0.5% |
| Synthalen K (4) | 0.7% |
| Triethanolamine | 0.6% |
| Water | qs 100% |

(1) isoeicosane sold by the company Bayer
(2) tridecyl trimellitate sold by the company Lipo Chemicals
(3) oxyethylenated polydimethylsiloxane comprising phosphate groups, sold by the company Phoenix
(4) polyacrylic acid sold by the company 3V

Composition B was applied to natural hair using a pump-dispenser bottle. After drying, it was observed that the lock was shiny, feels soft and looks pleasant.

### EXAMPLE 3

Composition C below was applied to a 5-gram lock of natural European chestnut-brown hair.

Composition C:

| | |
|---|---|
| Permethyl 102 A (1) | 5% |
| Liponate TDTM (2) | 5% |
| DC 245 Fluid (3) | 10% |
| DC2-5185C INT (4) | 2.5% |
| Water | qs 100% |

(1) isoeicosane sold by the company Bayer
(2) tridecyl trimellitate sold by the company Lipo Chemicals
(3) cyclopentadimethylsiloxane sold by the company Dow Corning
(4) oxyethylenated oxypropylenated polydimethylsiloxane sold by the company Dow Corning

Composition C was applied to natural hair using a pump-dispenser bottle. After drying, soft and shiny hair was obtained.

1. A cosmetic hair composition comprising, in a cosmetically acceptable medium, at least one tridecyl trimellitate and at least one isoeicosane.

2. The composition according to claim 1, wherein the tridecyl trimellitate is present in the composition in an amount ranging from 0.01% to 15% and the isoeicosane is present in the composition in an amount ranging from 0.01% to 15% by weight relative to the total weight of the composition.

3. The composition according to claim 2, wherein the tridecyl trimellitate is present in the composition in an amount ranging from 1% to 10% and the isoeicosane is present in the composition in an amount ranging from 0.05% to 20% by weight relative to the total weight of the composition.

4. The composition according to claim 1, wherein the tridecyl trimellitate and isoeicosane are present in a combined amount ranging from 0.05% to 20% by weight relative to the total weight of the composition.

5. The composition according to claim 4, wherein the tridecyl trimellitate and isoeicosane are present in a combined amount ranging from 5% to 10% by weight relative to the total weight of the composition.

6. The composition according to claim 1, wherein the tridecyl trimellitate/isoeicosane weight ratio ranges from 100 to 0.01.

7. The composition according to claim 6, wherein the tridecyl trimellitate/isoeicosane weight ratio ranges from 10 to 0.1

8. The Composition according to claim 7, wherein the tridecyl trimellitate/isoeicosane weight ratio ranges from 0.7 to 1.3.

9. The composition according to claim 1, wherein the composition further comprises at least one fixing polymer.

10. The composition according to claim 9, wherein the at least one fixing polymer is chosen from cationic, anionic, amphoteric and nonionic fixing polymers.

11. The composition according to claim 10, wherein the at least one fixing polymer is a mixture of fixing polymers.

12. The composition according to claim 10, wherein the cationic fixing polymer is chosen from acrylic esters, methacrylic esters, and amide homopolymers and copolymers comprising amine functional groups; cationic polysaccharides; quaternary copolymers of vinylpyrrolidone and of vinylimidazole; and chitosans.

13. The composition according to claim 10, wherein the anionic fixing polymer is chosen from acrylic and methacrylic acid homopolymers and copolymers and salts thereof; crotonic acid copolymers; C₄-C₈ monounsaturated carboxylic acid and anhydride copolymers; polyacrylamides comprising carboxylate groups; homopolymers and copolymers comprising sulfonic groups; anionic polyurethanes; and anionic grafted silicone polymers.

14. The composition according to claim 10, wherein the amphoteric fixing polymer is chosen from copolymers comprising acidic vinyl units and basic vinyl units, crosslinked and acylated polyamino amides, polymers comprising zwitterionic units, chitosan-based polymers, modified (C₁-C₅)alkyl vinyl ether/maleic anhydride copolymers, amphoteric polyurethanes and amphoteric grafted silicone polymers.

15. The composition according to claim 10, wherein the nonionic fixing polymer is chosen from polyalkyloxazolines, vinyl acetate homopolymers and copolymers, acrylic ester homopolymers and copolymers, acrylonitrile copolymers, styrene homopolymers and copolymers, polyamides, vinyllactam homopolymers other than vinylpyrrolidone homopolymers, vinyllactam copolymers, nonionic polyurethanes and nonionic grafted silicone polymers.

16. The composition according to claim 9, wherein the at least one fixing polymer is present in the composition in an amount ranging from 0.01% to 20% by weight relative to the total weight of the composition.

17. The composition according to claim 16, wherein the at least one fixing polymer is present in the composition in an amount ranging from 0.05% to 15% by weight relative to the total weight of the composition.

18. The composition according to claim 17, wherein the at least one fixing polymer is present in the composition in an amount ranging from 0.1% to 10% by weight relative to the total weight of the composition.

19. The composition according to claim 1, wherein the composition further comprises at least one additive chosen from nonionic, anionic, cationic, amphoteric and zwitterionic surfactants, fragrances, screening agents, preserving agents, proteins, vitamins, nonionic, anionic, cationic or zwitterionic polymers other than the fixing polymers featured in the preceding claims, mineral oils, plant or synthetic oils, thickening agents and any other additive conventionally used in cosmetic compositions, antidandruff agents, agents for combating hair loss, dyes, pigments, moisturizers and reducing agents.

20. A cosmetic hair process comprising applying to hair a composition comprising, in a cosmetically acceptable medium, at least one tridecyl trimellitate and at least one isoeicosane.

21. A process for giving hair sheen, comprising applying to the hair a composition comprising, in a cosmetically acceptable medium, at least one tridecyl trimellitate and at least one isoeicosane.
